(19) United States
(12) Patent Application Publication
(10) Pub. No.: US 2024/0115593 A1
(43) Pub. Date: Apr. 11, 2024

(54) METHISOPRINOL COMPOSITIONS

(71) Applicant: REENA PATEL, Mumbai, Maharashtra (IN)

(72) Inventors: Reena PATEL, Mumbai, Maharashtra (IN); Dinesh Shantilal PATEL, Mumbai, Maharashtra (IN); Sachin Dinesh PATEL, Mumbai, Maharashtra (IN); Shashikant Prabhudas KURANI, Mumbai, Maharashtra (IN); Milind Vinayak SATHE, Mumbai, Maharashtra (IN)

(21) Appl. No.: 18/256,754

(22) PCT Filed: Dec. 10, 2021
(86) PCT No.: PCT/IB2021/061559

§ 371 Date: Jun. 9, 2023

(30) Foreign Application Priority Data
Dec. 10, 2020 (IN) 202021053711

(51) Int. Cl. A61K 31/708; A61K 9/08; A61K 47/02; A61K 47/10; A61K 47/20

(52) U.S. Cl. CPC A61K 31/708; A61K 9/08; A61K 47/02; A61K 47/10; A61K 47/20

(57) ABSTRACT

The present invention provides a stable aqueous composition of Methisoprinol. In particular, it provides a stable aqueous injectable composition comprising 25 to 150 mg/ml of Methisoprinol, at least one alcohol, and at least one excipient selected from pH modifier or buffer, surfactant, solvents, co-solvent, preservative, antioxidants, chelating agent and a tonicity modifier. The alcohol is present in proportion up to 40% w/v, preferably up to 30% w/v. The invention also provides methods of preparation of such composition and uses thereof in the treatment or prevention of viral diseases and immune suppressed states.

### FIELD OF THE INVENTION

The present invention relates to stable aqueous compositions of Methisoprinol. In particular, it relates to a stable aqueous injectable composition comprising 25 to 150 mg/ml of Methisoprinol, at least one alcohol, and at least one excipient selected from pH modifier or buffer, surfactant, solvents, co-solvent, preservative, antioxidants, chelating agent and a tonicity modifier. The alcohol is present in proportion up to 40% w/v, preferably up to 30% w/v. The invention also relates to methods of preparation of such compositions and to uses thereof in the treatment or prevention of viral diseases and immune suppressed states.

### BACKGROUND OF INVENTION

Methisoprinol, also known as Inosine pranobex, isoprinosine or inosine dimepranolacedoben, is a combination of inosine, acetamidobenzoic acid and dimethylaminoisopropanol. Methisoprinol is represented by the following structure.

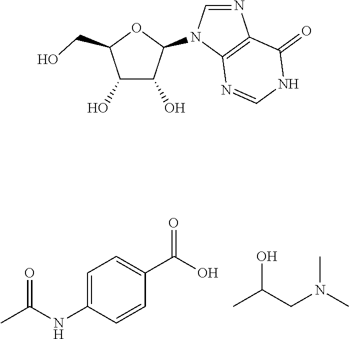

Methisoprinol as a drug was initially authorized in 1971 and is currently marketed in more than 70 countries worldwide for the treatment of viral diseases, including subacute sclerosingpanencephalitis (SSPE), herpes simplex virus (HSV) and varicella infections, human papilloma virus (HPV), cytomegalovirus and Epstein-Barr virus infections, acute viral respiratory infections, measles, and immunosuppressed states.

Methisoprinol is supplied for long time in the form of solid dosage forms such as Tablets or capsules. While preparing aqueous formulations of Methisoprinol, researchers faced certain difficulties, for instance, degradation of actives. Actives when presented in liquid form often get degraded in the presence of moisture or other ingredients. Additionally, it is known that a solution of Methisoprinol has a bitter taste and is unstable during storage. During prolonged storage, impurities are formed. To make the composition stable and nullify the bitter taste, generally sugar is added which makes the composition unsuitable for diabetic patients.

Further, from oral compositions, Methisoprinol takes some time to reach required blood levels which is undesirable in acute conditions. For instance, when a patient is in severe condition or is physically unable to consume oral dosage form, it is necessary to administer liquid injectable dosage form. However, at present there is a lack of injectable compositions of Methisoprinol for humans.

Conventionally formulated Methisoprinol injections are limited to intramuscular administration. This limitation is not as a consequence of intravenous safety profile, but principally due to physico-chemical properties of the drug. Poor aqueous solubility of Methisoprinol has a particularly high tendency to crystallize from aqueous and organic solutions.

US20160166683A1 mentions Isoprinosine but does not describe the process to prepare a stable injectable composition or product of Isoprinosine.

US20050148408 describes an invention wherein a dried form of a primary aqueous solubilized bile acid formulation comprises: (a) a first material selected from the group consisting of a bile acid, an aqueous soluble derivative of a bile acid, a bile acid salt, a bile acid conjugated with an amine by an amide linkage, and combinations thereof, and (b) an aqueous soluble starch conversion product; wherein the first material and the aqueous soluble starch conversion product both remain in solution for all pH values of the solution within a selected range of pH values. Although the patent application mentions Isoprinosine, it does not give an example or teach stable injectable composition of Isoprinosine. Further, presence of bile salt is an essential feature of formulation disclosed in this invention. Sometimes consumption of bile salts is not tolerated by individuals.

US20060142241 although mentions Isoprinosine it does not teach stable injectable composition of Isoprinosine and the invention resides in making of a solution of bile salt.

US20130064841 describes a polypeptide immunogen comprising (A) a mimetic peptide comprised of (i) the amino acid sequence of a progastrin or a N-terminal and/or C-terminal processed species of a progastrin joined to (ii) a 7 amino-acid spacer and (B) an immunogenic carrier coupled to said mimetic peptide. It makes a mention of Isoprinosine as an adjuvant but does not teach stable injectable composition of Isoprinosine.

WO03013544 relates to an antiviral medicament for use in controlling white spot syndrome in shrimp. The invention consists in using known molecules, the acyclovir antiviral and the Methisoprinol immunostimulant mixed in a suitable formula and using a water-insoluble vehicle (calcium carbonate) in order to treat white spot syndrome.

IL75915 is related to Methisoprinol tablets. IT1270832 describes a pharmaceutical composition containing 2-amino-4,6-dichloropyrimidine and Methisoprinol in equal parts by weight as active ingredients is described; the said composition is useful as an antiviral agent.

US20020031558 is a publication describing a method for treating gastritis and peptic ulcer disease comprising: (a) administration of an oral liquid dosage form comprising: (i) a first material selected from the group consisting of a bile acid, an aqueous soluble derivative of a bile acid, a bile acid salt, a bile acid conjugated with an amine by an amide linkage, and combinations thereof; (ii) a second material selected from the group consisting of an aqueous soluble starch conversion product and an aqueous soluble non-starch polysaccharide; and (iii) water, wherein the first and second materials both remain in solution for all pH values of the solution within a selected range of pH values. This invention makes mention of Isoprinosine like several other compounds as an additional compound to be added in composition of the invention. But entire description is silent about how to prepare a stable injectable composition of Isoprinosine.

WO2019226058 teaches a medicinal product containing improved stability water solution of Methisoprinol, stable at a temperature range between +2° C. and +8 C., characterized in that it contains 1-dimethylaminopropan-2-ol 4 acetanidobenzoate in the amount from more than 2% to 14% of weight in relation to the amount of 1-dimethylaminopropan-2-ol 4-acetamidobenzoate present in dissolved Methisoprinol. The invention resides in incorporating additional 1-dimethylaminopropan-2-ol 4-acetamidobenzoate salt in an amount above 2% in relation to the amount of salt contained in dissolved Methisoprinol. It was demonstrated that if additional quantities are not added then Methisoprinol crystallize out after 6 days when stored at temperature of +20° C. to +2° C. It mentions that solution comprising 20% (w/v) of Methisoprinol, 0.5% (w/v) of phenol as a preservative, and water for injection up to 100%, the solution was once known as the veterinary medicinal product Isoprivet 20% for injections but it exhibited crystallization when stored at temperature of +20° C. to +2° C.

Incorporating additional quantities of 1-dimethylaminopropan-2-ol 4-acetamidobenzoate salt would render the active out of pharmacopoeial or standard specifications and rendering it unfit for administration. WO2016003313A1 describes a pharmaceutical composition for oral administration, comprising Methisoprinol, methyl-p-hydroxybenzoate, propyl-p-hydroxybenzoate, sucrose or maltose, glycerin, citrus flavoring and purified water. However, composition of this document contains sugar and hence is not recommended for diabetics. Sucrose or maltose 585-715 mg/ml is necessary as preservative and without that syrup will not be stable. Methisoprinol will degrade fast. In addition it requires presence of several other preservatives such as methyl-p-hydroxybenzoate, propyl-p-hydroxybenzoate. Thus the composition has several other chemicals in it which are undesirable. It is generally known that individuals above the age of 45 are prone for disturbances in glucose metabolism or diabetes across the globe. The document does not teach injectable composition or does not provide hint or motivation to prepare sugar less or maltose less aqueous injectable composition.

EP2337567 describes a veterinary oral composition comprising Methisoprinol. The patent document mentions that Methisoprinol is preferably dissolved in water or is suspended in polyhydric alcohol such as propylene glycol or sorbitol which are present as high as 70% w/v. It advises use of phenol as preservative. The document is related to veterinary composition for oral administration. It does not teach an injectable composition of Methisoprinol suitable for humans. Technique of developing injectable composition is dissimilar to that of developing composition for oral veterinary use. Although it claims veterinary oral composition it is devoid of any stability data. Ingredients like guar gum has been used which cannot be used for injectable composition. Use of huge quantities of alcohol is another drawback of this composition. Regulatory guidelines do not allow use of so high quantities of propylene glycol. Inactive ingredient database stipulates use of 41.6% w/v of propylene glycol for intramuscular injection solution. For intravenous solution it is just 5% w/v. Thus the invention claimed in this patent document provides formulae that are not consistent with inactive ingredient database guidelines. The invention in this patent document teaches away from preparing a stable aqueous injectable composition. Invention in the present application does not use so huge quantities of alcohol or does not use sugar guar gum for stabilization of composition yet provides a stable composition comprising Methisoprinol that can be administered by injection or parenteral route.

The oral compositions available till date comprise excessive quantities of chemicals such as sugar (about 60% w/v), propylene glycol (about 70% w/v), sorbitol (about 70% w/v), guar gum, celluloses. It is always better to use ingredients other than actives in small quantities or to avoid using them. Use of huge quantities of ingredients other than actives limits utility of the compositions of prior art. This is one of the reasons for lack of sufficient literature directed to injectable compositions or stability data of active or injectable compositions. Available prior art is limited only to veterinary oral or oral compositions of Methisoprinol.

The scrupulous study of prior art points to glaring limitations of status of prior art and challenges it failed to overcome. Firstly, Methisoprinol is poorly soluble in non-aqueous solvents and has poor solubility in water. Prior art is devoid of stable injectable compositions of Methisoprinol having concentration upto 150 mg/ml. There is hardly any data on multiple solvent systems employed to formulate a stable injectable composition of Methisoprinol. Possibly due to poor stability of liquid compositions and more specifically of aqueous compositions, liquid injectable compositions of Methisoprinol are not available in market.

The lack of stable liquid injectable composition indicates presence of some irresolvable deadlock. Known liquid injectable compositions had inherent problems such as crystallization and degradation of actives. Thus prior art failed to provide stable safe liquid injectable composition of desired concentration of Methisoprinol. Another biggest challenge was susceptibility of Methisoprinol to oxidation or deamination or such other chemical degradations. As quantity of Methisoprinol per ml increases, possibility of fast degradation and crystallization also increases. Thus preparing a stable injectable composition with higher quantity of Methisoprinol per ml is a challenge. Stabilizing aqueous liquid composition of Methisoprinol is a greater challenge.

Till date no injectable composition of Methisoprinol seems to be available for human. Stabilizing Methisoprinol in aqueous environment is a challenge. In other words, there is an unmet need of stable injectable composition of Methisoprinol that remains clear and free from crystallization defect at lower temperatures and degradation in aqueous environment.

The present invention solves the problems of prior art and provides stable liquid injectable compositions of Methisoprinol. The present invention provides therapeutically effective amount of Methisoprinol in as aqueous composition suitable to be administered by multiple routes.

### OBJECTS OF THE INVENTION

It is an object of the present invention to provide a stable liquid composition of Methisoprinol.

It is another object of the invention to provide a liquid composition of Methisoprinol which is free from crystallization defect at lower temperatures and degradation in aqueous environment.

It is yet another object of the invention to provide an injectable composition of Methisoprinol for humans.

It is a further object of the invention to provide a method of preparation of stable injectable liquid composition of Methisoprinol suitable for humans.

It is a yet another object of the invention to provide a method of treatment or prevention of viral diseases and immune suppressed states.

### SUMMARY OF THE INVENTION

The inventors have unexpectedly discovered that a stable liquid composition of Methisoprinol can be formulated with particular amounts of specific alcohols. The inventors unexpectedly employed less or extremely low quantity of alcohol to surprisingly arrive at a stable injectable compositions of Methisoprinol.

Present invention employs from 10% w/v to 40% w/v of alcohol, preferably between 20% w/v to 30% w/v of alcohol to stabilize Methisoprinol. The composition is stable at 40° C.±2° C. and 75%±5% RH for 6 months. It is free from crystallization defect at lower temperatures and degradation in aqueous environment. The composition is fit for parenteral administration. The invention provides a stable aqueous composition comprising therapeutically effective amount of Methisoprinol suitable to be administered by multiple routes of administration to humans.

The present invention provides a stable liquid injectable composition comprising Methisoprinol in an amount from 25 mg/ml to 150 mg/ml, at least one alcohol in an amount from 20% w/v to 40% w/v and at least one excipient selected from pH modifier or buffer, surfactant, solvents, co-solvent, preservative, antioxidants, chelating agent and tonicity modifier.

In particular, the present invention provides stable, clear injectable composition comprising 100 mg/ml to 150 mg/ml Methisoprinol, 30% w/v to 40% w/v alcohol and at least one excipient selected from pH modifier or buffer, surfactant, solvents, co-solvent, preservative, antioxidants, chelating agent and tonicity modifier.

In one particular embodiment, the present invention provides stable, clear injectable composition comprising 100 mg/ml Methisoprinol, 20% w/v propylene glycol and 20% w/v glycerin, 0.25% w/v monothioglycerol and 1% w/v sodium chloride.

In an embodiment, the present invention provides a method of preparation of stable aqueous composition comprising Methisoprinol.

In yet another embodiment, the present invention provides a use of the composition of methisoprinol in manufacture of a medicament for treatment and prevention of viral disease and immune-suppressed states.

In a further embodiment, the present invention provides a method of treatment or prevention of viral disease and immune-suppressed states comprising administering to a patient in need thereof, a therapeutically effective amount of the composition of present invention.

### Brief Description of the Embodiments

The invention provides a stable injectable composition comprising Methisoprinol in an amount up to 150 mg/ml. In particular, the invention provides a stable injectable composition comprising Methisoprinol in an amount from 25 mg/ml to 150 mg/ml, at least one alcohol in an amount from 20% w/v to 40% w/v and at least one excipient selected from pH modifier or buffer, surfactant, solvents, co-solvent, preservative, antioxidants, chelating agent and tonicity modifier. More particularly, the present invention provides a stable injectable composition comprising Methisoprinol in an amount from 100 mg/ml to 150 mg/ml, at least one alcohol in an amount from 30% w/v to 40% w/v and at least one excipient selected from pH modifier or buffer, surfactant, solvents, co-solvent, preservative, antioxidants, chelating agent and tonicity modifier.

In an embodiment, the composition comprises Methisoprinol in an amount of 150 mg/ml. In another embodiment, the composition comprises Methisoprinol in an amount of 125 mg/ml. In yet another embodiment, the composition comprises Methisoprinol in an amount of 100 mg/ml.

The Methisoprinol is dissolved in alcohol. In an embodiment alcohol is present in an amount of 20% w/v. In another embodiment, alcohol is present in an amount of 30% w/v. In yet another embodiment, alcohol is present in an amount of 40% w/v.

The alcohol is selected from monohydric such as ethyl alcohol, benzyl alcohol or polyhydric alcohol such as glycerin, propylene glycol, monothioglycerol, sorbitol or combination thereof. In one embodiment, the composition comprises propylene glycol and glycerin in a ratio from 0.0:40 to 3:1. In particular embodiment propylene glycol and glycerin are present in a ratio 0.0:40 to 1:1. In another embodiment, the composition comprises ethyl alcohol and glycerin in a ratio of 0:10 to 10:0, preferably 1:1. In yet another embodiment, the composition comprises ethyl alcohol and propylene glycol in the ratio from 1:10 to 10:1.

In one embodiment, the composition comprises 1% w/v to 40% w/v glycerin. In particular embodiment, the composition comprises 15% w/v to 40% w/v glycerin. In another embodiment, the composition comprises 10% w/v to 20% w/v propylene glycol. In yet another embodiment, ethyl alcohol is present in in an amount between 10% w/v to 20% w/v.

In another embodiment, the composition comprises 20% w/v propylene glycol and 20% w/v glycerin. In yet another embodiment, the composition comprises 15% w/v propylene glycol and 15% w/v glycerin.

In another embodiment, the composition comprises propylene glycol and glycerin in a ratio from 0.0:40 to 3:1. In yet another embodiment, the composition comprises propylene glycol and glycerin in a ratio preferably from 0.0:40 to 1:1.

In an embodiment, the composition comprises ethyl alcohol and glycerin in a ratio of 0:10 to 10:0, preferably 1:1. In yet another embodiment, the composition comprises ethyl alcohol and propylene glycol in the ratio from 1:10 to 10:1.

In a particular embodiment, the composition comprises 100 mg/ml Methisoprinol, 20% w/v propylene glycol and 20% w/v glycerin. In another particular embodiment, the composition comprises 100 mg/ml Methisoprinol, 15% w/v propylene glycol and 15% w/v glycerin. In yet another particular embodiment, the composition comprises 150 mg/ml Methisoprinol, 20% w/v propylene glycol and 20% w/v glycerin.

In still another particular embodiment, the composition comprises 100 mg/ml Methisoprinol, 20% w/v propylene glycol and 20% w/v glycerin, 0.25% w/v monothioglycerol and 1% w/v sodium chloride.

In a particular embodiment, the composition comprises 125 mg/ml Methisoprinol and 20% w/v to 40% w/v glycerin.

In a particular embodiment, the composition comprises monothioglycerol in an amount from 0.25% w/v to 1% w/v and sodium chloride upto 1% w/v.

In a particular embodiment the present invention provides a stable, clear, aqueous injectable composition comprising 100 mg/ml Methisoprinol, 20% w/v propylene glycol and 20% w/v glycerin, 0.25% w/v monothioglycerol and 1% w/v sodium chloride.

In yet another embodiment, the composition is an injectable composition and is suitable for parenteral administration.

In yet another embodiment, the composition has viscosity not more than 10 cps. In yet another embodiment, the composition has pH from 6.0 to 7.5.

In an embodiment, the composition of present invention is suitable for use in treatment of disease or disorder selected from the group consisting of genital warts, subacute sclerosingpanencephalitis (SSPE), herpes simplex virus (HSV) and varicella infections, human papilloma virus (HPV), cytomegalovirus and Epstein-Barr virus infections, acute viral respiratory infections, measles virus, exanthematous viral infections like chickenpox, measles, bronchitis, common cold (Rhinopharyngitis) and immune-suppressed states.

In an embodiment, the present invention provides a method of preparing a stable aqueous composition comprising Methisoprinol, wherein the method comprises:
a) weighing required quantities of all raw materials by suitable means;
b) collecting sufficient quantity of water in jacketed manufacturing tank and cooling it to about 25±2° C. with continuous stirring and nitrogen sparging in closed condition;
c) transferring about ⅔^{rd} of water for injection from jacketed manufacturing tank referred in step b) to separate SS vessel and starting nitrogen bubbling while keeping remaining quantity of water aside for volume adjustment;
d) adding required quantity of sodium chloride in jacketed manufacturing tank referred in b) under stirring and nitrogen bubbling followed by stirring for about 5 minutes with lid closed;
e) adding required quantity of alcohol into the jacketed manufacturing tank referred in b) under stirring and nitrogen bubbling followed by optionally adding one or more stabilizers under continuous stirring till a clear solution is obtained;
f) adding required quantity of Methisoprinol to clear solution obtained in step (e) under stirring and nitrogen bubbling while keeping the tank closed under continuous stirring to obtain a clear solution;
g) adjusting pH of the clear solution obtained in step (f) to about 6.0 to 7.5;
h) adding water to make final volume while continuously stirring for about 30 minutes with nitrogen bubbling in a closed tank until clear solution was obtained;
i) closing all openings of the jacketed manufacturing tank blanketed with nitrogen bubbling until filtration is complete;
j) optionally, packing the solution prepared in step (i) after filtration in suitable containers.

In an embodiment, the present invention provides use of the composition of present invention in the manufacture of a medicament for treatment and prevention of viral disease and immune-suppressed states. In another embodiment, the present invention provides use of the composition of present invention in the manufacture of a medicament for treatment and prevention of viral disease, wherein the viral disease is selected from the group consisting of genital warts, subacute sclerosingpanencephalitis (SSPE), herpes simplex virus (HSV) and varicella infections, human papilloma virus (HPV), cytomegalovirus and Epstein-Barr virus infections, acute viral respiratory infections, measles virus, exanthematous viral infections like chickenpox, measles, bronchitis, common cold (Rhinopharyngitis).

In an embodiment, the present invention provides a method of treatment or prevention of viral disease and immune-suppressed states comprising administering to a patient in need thereof, a therapeutically effective amount of the composition of present invention. In a further embodiment, the present invention provides a method of treatment or prevention of viral disease, wherein the viral disease is selected from the group consisting of genital warts, subacute sclerosingpanencephalitis (SSPE), herpes simplex virus (HSV) and varicella infections, human papilloma virus (HPV), cytomegalovirus and Epstein-Barr virus infections, acute viral respiratory infections, exanthematous viral infections like chickenpox, measles, bronchitis, common cold (rhinopharyngitis).

### DETAILED DESCRIPTION OF THE INVENTION

The present invention provides a stable aqueous injectable composition comprising Methisoprinol in an amount upto 150 mg/ml, at least one alcohol in an amount upto 40% w/v and at least one excipient selected from pH modifier or buffer, surfactant, solvents, co-solvent, preservative, antioxidants, chelating agent and tonicity modifier.

The aqueous compositions of Methisoprinol are known to be unstable, it was surprisingly found that aqueous compositions of Methisoprinol are stable in presence of at least one alcohol, provided, alcohol is present in stipulated amounts. The amount of alcohol used in the present invention is substantially less than amount used in prior art compositions which were unfit for injectable administration.

The alcohol can be monohydric or polyhydric. A mixture of monohydric and polyhydric alcohol when incorporated in specific proportions also results into stable aqueous compositions of Methisoprinol fit for parenteral applications. An alcohol other than monohydric alcohol is to be understood as polyhydric alcohol for the purposes of the present invention. Therefore, alcohol mentioned hereinafter is to be understood and interpreted as alcohol selected from monohydric alcohol or polyhydric alcohol or mixture thereof.

The composition of present inventions comprises 25-150 mg/ml of Methisoprinol and alcohol in an amount from 20% w/v to 40% w/v. The alcohol is selected from a monohydric alcohol or a polyhydric alcohol or a combination thereof. The monohydric alcohol is selected from but not limited to ethyl alcohol and benzyl alcohol. The polyhydric alcohol is selected from but not limited to glycerin, propylene glycol, monothioglycerol, sorbitol or combination thereof.

The compositions are stable for at least 6 months at 40° C./75% RH, 25° C./60% RH, 30° C./75% RH, 30° C./65% RH (as per standard ICH guidelines).

In another aspect the invention provides method of preparation of injectable composition of Methisoprinol.

Compositions of the present invention are prepared by the method comprising the steps of
a) weighing required quantities of all raw materials by suitable means;
b) collecting sufficient quantity of water in jacketed manufacturing tank and cooling it to about 25±2° C. with continuous stirring and nitrogen sparging in closed condition;
c) transferring about ⅔rd of water for injection from jacketed manufacturing tank referred in step b) to separate SS vessel and starting nitrogen bubbling while keeping remaining quantity of water aside for volume adjustment;
d) adding required quantity of sodium chloride in jacketed manufacturing tank referred in b) under stirring and nitrogen bubbling followed by stirring for about 5 minutes with lid closed;
e) adding required quantity of alcohol into the jacketed manufacturing tank referred in b) under stirring and nitrogen bubbling followed by optionally adding one or more stabilizers under continuous stirring till a clear solution is obtained;
f) adding required quantity of Methisoprinol to clear solution obtained in step (e) under stirring and nitrogen bubbling while keeping the tank closed under continuous stirring to obtain a clear solution;
g) adjusting pH of the clear solution obtained in step (f) to about 6.0 to 7.5;
h) adding water to make final volume while continuously stirring for about 30 minutes with nitrogen bubbling in a closed tank until clear solution was obtained;
i) closing all openings of the jacketed manufacturing tank blanketed with nitrogen bubbling until filtration is complete;
j) optionally, packing the solution prepared in step (i) after filtration in suitable containers.

An embodiment of the method of present invention is illustrated herein below.
a) required quantities of all raw materials were weighed by suitable means.
b) in jacketed manufacturing tank about 30 liters of water was collected for injection and cooled to about 25±2° C., with continuous stirring and nitrogen bubbling/sparging, in closed condition.
c) About 20 liters of water was transferred for injection from the jacketed manufacturing tank to separate SS vessel and started nitrogen bubbling/sparging. The remaining quantity of water is kept aside for volume adjustment.
d) required quantity of sodium chloride was added in the above jacketed manufacturing tank under stirring and nitrogen bubbling/sparging. Stirring was performed for about 5 minutes with lid closed.
e) required quantity of alcohol was added into the jacketed manufacturing tank under stirring and nitrogen bubbling/sparging. Optionally, one or more stabilizers can also be added at this stage. Stirring was performed till a clear solution is obtained.
f) required quantity of Methisoprinol was added into jacketed manufacturing tank under stirring and nitrogen bubbling/sparging. The tank was closed and stirring was continued till a clear solution was obtained.
g) pH of the clear solution obtained in above step was adjusted to about 6.0 to 7.5.
h) The final volume was made upto 25 liters by adding water for injection from the SS container. The solution was stirred for 30 minutes with nitrogen bubbling/sparging in a closed tank. The stirring was stopped after clear solution was formed.
i) All opening of jacketed manufacturing tank were closed. The manufacturing tank was blanketed with nitrogen bubbling till filtration was complete.

The process of filtration performed is as follows:

### First Filtration

1. The sterile filter assembly was arranged and the filter was wetted with water for injection.
2. The pre bubble point test was performed for 0.22μ Nylon 66 membrane filters.
3. The sterilized assembly was arranged for filtration. The pressure vessel was pre-flushed with sterile nitrogen prior to start of filtration.
4. Filtration was carried through 2.0μ and 0.22μ membrane filter with help of filtered nitrogen.
5. After completion of filtration, the post bubble point test was performed.

### Second Filtration Procedure (Before Filling)

1. The sterile filter assembly was arranged and the filter was wetted with water for injection.
2. The pre bubble point test was performed for 0.22μ Nylon 66 membrane filters.
3. The sterilized assembly for filtration was arranged. The pressure vessel was pre flushed with sterile nitrogen prior to start of filtration.
4. Filtration through 0.22μ membrane filter was carried with help of filtered nitrogen and solution was collected in filling vessel.
5. The filtration was started along with continuous filling activity.

After completion of filtration, the post bubble point test was performed. The compositions thus prepared are stable. Compositions of the present invention when kept in vials without sterilization are also stable. Thus it opens an avenue for administration of Methisoprinol aqueous liquid compositions for non-injectable routes such as oral administration.

In one embodiment, composition comprising propylene glycol, glycerin and monothioglycerol is prepared. In another embodiment, composition is prepared without propylene glycol. In yet another embodiment composition is prepared without monothioglycerol.

In one embodiment, a composition comprising 125 mg per ml Methisoprinol, 20% w/v propylene glycol and 20% w/v glycerin, 0.25% w/v monothioglycerol, and 0.9% w/v sodium chloride is prepared, In other embodiment, composition is prepared with 40% w/v glycerin and no propylene glycol is incorporated.

In the similar manner composition comprising 150 mg/ml Methisoprinol, 0.25% w/v monothioglycerol, 0.9% w/v sodium chloride and 20% w/v glycerin is prepared. In another embodiment, amount of glycerin is increased to 40% w/v. Surprisingly it is observed that a stable aqueous composition comprising Methisoprinol upto 150 mg/ml can be prepared with 40% w/v glycerin.

The preferred alcohols used in the present invention are selected from glycerin, propylene glycol, monothioglycerol and ethyl alcohol. The amount of alcohol used varies with the amount of Methisoprinol. The amount of glycerin used varies between %1 w/v to 40% w/v. In preferred embodiments, the composition comprises 5% w/v to 40% w/v of glycerin. In more preferred embodiments, composition comprises 10% w/v to 20% w/v of glycerin. Examples 1 and 4 provide compositions comprising 15% w/v of glycerin whereas some other examples illustrate use of 20% w/v of glycerin.

The amount of glycerin can be adjusted with inclusion of appropriate quantities of other alcohols selected from, but not limited to, propylene glycol, monothioglycerol, and ethyl alcohol. Thus a stable composition can be prepared by comprising at least 10% w/v of glycerin. Amount of glycerin required also depends on quantity of Methisoprinol to be stabilized per ml of aqueous environment.

In another embodiment, propylene glycol is incorporated in an amount from 0% w/v to about 20% w/v. Stable compositions can be prepared without propylene glycol. In preferred embodiment, propylene glycol when used in an amount from 1% w/v to about 20% gives good results. In more preferred embodiment, the amount of propylene glycol used is between 5% w/v to 15% w/v. Examples 1 and 4 provide compositions comprising 15% w/v of propylene glycol whereas example 9 provides composition with 10% w/v of propylene glycol. Example 7 provides composition comprising 20% w/v of propylene glycol. In some embodiments ratio of propylene glycol to glycerin used in the compositions is 3:1. The preferred ratio of propylene glycol to glycerin is 0:40 to 1:1 (example 7). More preferably, the ratio of propylene glycol to glycerin used is 1:1. In some embodiments, total amount of propylene glycol and glycerin used is 30% w/v. In other embodiments, the total amount of propylene glycol and glycerin used is 40% w/v.

In yet another embodiment, stable liquid aqueous compositions of Methisoprinol for parenteral administration are prepared using ethyl alcohol with or without glycerin. Example 9 provides composition comprising ethyl alcohol. In preferred embodiment, the composition comprises 20% w/v of ethyl alcohol. Due to limitations on amount of ethyl alcohol that can enter the body, the total amount of ethyl alcohol may be used is 20% w/v. One may incorporate more than 20% w/v of ethyl alcohol if the total quantum of alcohol that shall enter the body is within the limits allowed by regulatory guidelines. Use of larger quantities of ethyl alcohol is generally not allowed for potential health hazards. Additionally, for pediatric use, it is undesirable to use huge quantities of ethyl alcohol. In one embodiment, the composition comprises ethyl alcohol to propylene glycol in the ratio of 1:10 to 10:1. Example 9 provides composition comprising ethyl alcohol and propylene glycol in a ratio of 1:1. In similar manner, person skilled in the art will be able to prepare satisfactory stable compositions wherein ratio of ethyl alcohol to propylene glycol in the ratio of 1:10 to 10:1 with or without glycerin.

In other embodiment, incorporation of ethyl alcohol with glycerin results into stable compositions. In one embodiment, composition comprises ethyl alcohol to glycerin in a ratio of 0:10 to 10:0. In preferred embodiments ratio of ethyl alcohol to glycerin is 1:1. These compositions optionally comprise propylene glycol with ethyl alcohol. As example and procedure to prepare stable compositions using propylene glycol and alcohol is self-explanatory, one can easily prepare compositions in which propylene glycol is replaced by glycerin either partially or fully. Various possible embodiments of such compositions, in context of disclosure, can be easily prepared, however, for the reasons of brevity and conciseness of specification have not been illustrated herein.

It was observed that when total amount of alcohol is kept less than or equal to 40% w/v, preferably upto or less than 30% w/v, stable aqueous injectable compositions comprising Methisoprinol upto 150 mg/ml can be prepared. A person skilled in the art knows that ethyl alcohol, propylene glycol, monothioglycerol, glycerin, benzyl alcohol are all alcohols. Thus unlike prior art compositions, compositions of the present invention are aqueous stable injectable compositions comprising Methisoprinol upto 150 mg/ml and yet they do not contain huge quantities of alcohol. The compositions comprise either monohydric alcohols or polyhydric alcohols or combination thereof. Total amount of alcohol in compositions of the present invention do not increase more than 40% w/v irrespective of whether the alcohol is used singly or in combination.

In some embodiments, the composition comprises stabilizers. Sodium metabisulphite can be used to stabilize compositions of Methisoprinol. Sodium metabisulphite is known in the art for injectable compositions. In the present invention, monothioglycerol is preferred over sodium metabisulphite, monothioglycerol gives better stabilization than sodium metabisulphite.

Similarly compositions of the present invention prepared and subjected to sterilization by filtration were subjected to stability study at conditions as mentioned here before and were found to be stable.

In some embodiments, the composition comprises acetate buffer. It also acts as a solubilizer. It may be used individually or in combination with other ingredients like povidone, polysorbates, and glycine and alike.

It is observed that when 5% to 40% w/v of glycerin is incorporated, methisoprinol can be stabilized in aqueous environment. Stabilization means solubilizing Methisoprinol upto 150 mg/ml and preventing its crystallization when stored under prescribed stability testing conditions. Stabilization also means ensuring that active content of Methisoprinol remains within prescribed limits at the specific testing intervals as expressed in stability protocol under storage conditions as described in stability testing protocol based on ICH guidelines (Example 3). Stabilization means product conforms to prescribed specifications when stored under prescribed storage conditions. Stabilization also means that if at all any crystals are formed they go into solution or quickly dissolve when container is stirred or shaken. Needless to state that compositions do not produce impurities beyond specified limits when stored under storage condition as prescribed in stability protocol (Example 3).

In another aspect, the present invention provides a compositions comprising Methisoprinol in a concentration of upto 150 mg/ml for parenteral administration through IM/IV/infusion route and using the same liquid with suitable modification for use in ocular, dermal, otic, oral and for other preparations.

The viscosity of compositions provided in the present invention remains below 10 cps, preferably below 7 cps and more preferably below 5 cps. Thus, the compositions of the present invention are easily syringeable and can be administered with ease into tissues by health workers, thereby causing less pain and less pressure.

Compositions comprising 25 mg, 50 mg, 75 mg and 100 mg of Methisoprinol can be prepared in same manner as that of 150 mg/ml of Methisoprinol. For illustration, examples are restricted to compositions comprising 100 mg, 125 mg and 150 mg of Methisoprinol. However, compositions comprising lower amount of Methisoprinol viz., 25 mg, 50 mg, 75 mg and 100 mg also fall within the ambit of present invention and can be prepared in the same manner.

The composition comprises water as a solvent. The composition may comprise co-solvents other than alcohols. In some embodiments, the co-solvent is selected from but not limited to povidones like PVP K-12 upto 0.9%, benzyl benzoate upto 50%, castor oil upto 29%, and dimethylacetamide upto 33%. In some other embodiments the co-solvents are selected from, N-methyl-2-pyrrolidone, diethanolamine, L-arginine, peanut oil, poppy seed oil, safflower oil, sesame oil, soybean oil, vegetable oil, or any combination thereof. In yet another embodiments, compositions also comprise sesame oil, soybean oil and solvents alike. The peanut oil, poppy seed oil, safflower oil, sesame oil, soybean oil and vegetable oil are available commercially.

In some embodiments, the composition comprises antimicrobial preservatives selected from but not limited to benzethonium chloride 0.01%, benzyl alcohol upto 2%, benzalkonium chloride 0.02%, chlorobutanol 2.5 to 5%, m-cresol 0.1% to 0.3%, parabens like methylparaben, propyl paraben upto 1%, phenol upto 0.45%. Some more excipients like 2-phenoxyethanol, phenyl mercuric nitrate, thimerosal and excipients alike may be used in the formulation.

In another embodiment, composition may comprise chelating agent to chelate traces of metallic impurity. Chelating agents used are but not limited to disodium EDTA, sodium EDTA, Calcium disodium EDTA 0.2%, versetamide 2.54%, calteridol calcium 0.023% and ingredients alike.

In yet another embodiment, the composition comprises surfactants and co-surfactants selected from but not limited to polysorbates like polysorbate 80 up to 12%, polysorbate 20 upto 12%. In another aspect formulation may comprise other surfactants lecithin, polyoxyethylene-polyoxypropylene copolymers and like.

In still another embodiment, the composition comprises antioxidants selected from but not limited to sodium sulfite upto 0.2%, sodium bisulfite 0.1% to 1.6%, butylated hydroxy toluene 0.002% to 0.03%, cysteine hydrochloride 0.1%, potassium metabisulfite 0.1%, methionine 0.01% to 0.3% and monothioglycerol upto 1%. Antioxidants used in the formulation may be also selected from group of ascorbyl palmitate, ascorbate, dithionite sodium, genticic acid, genticic acid ethanolamine, propyl gallate, alpha tocopherol, sodium thioglycolate, glutathione formaldehyde sulfoxylate sodium and alike.

The pH is critical for maintaining stability of the Methisoprinol injection hence pH is maintained from 6.0 to 7.5, preferably from 6.0 to 7.0, more preferably 6.2 to 6.8 using suitable buffering agents. The buffering agents may be selected from, but not limited to alkali metal hydroxides like sodium hydroxide, potassium hydroxide, sodium citrate, sodium phosphate salts like monosodium phosphate salt or disodium phosphate salt, potassium phosphate salts like mono or di potassium phosphate salt, sodium acetate, glycine, lysine, meglumine, methanesulfonic acid etc. In another aspect the formulation may comprise buffering agents and pH adjusting agents which are maleic acid, sodium carbonate, sodium bicarbonate, citric acid, sodium citrate, disodium citrate, trisodium citrate, ammonium sulfate, ammonium hydroxide, sodium, potassium or ammonium salt of a weak acid, arginine, aspartic acid, benzene sulfonic acid, monoethanolamine, sodium succcinate, disodium succcinate, sodium tartarate, phosphate buffers,tris-(hydroxymethyl)-aminomethane, Tris base-65, Tris acetate, TrisHCl-65, hydrochloric acid, boric acid, citric acid, acetic acid, phosphoric acid, succinic acid with suitable salts and alike or, ammonium hydroxide, magnesium oxide, calcium carbonate, magnesium carbonate, malic acid, potassium citrate, sodium phosphate, lactic acid, gluconic acid, tartaric acid, fumaric acid, diethanolamine, monoethanolamine, triethanolamine, or any combination thereof. The stable aqueous injectable solution may comprise one or more pH adjusting agents in an amount to provide pH of the solution between about 6 and about 7. Buffers are used in sufficient quantity to maintain appropriate pH value during storage. Acetate buffer is used in some embodiments. Any substance that helps in maintaining pH of the composition in the range of 6-7 falls within the ambit of the invention.

In another embodiment Monothioglycerol act as antioxidant and it also possess preservative activity.

In the compositions provided in the present invention, water is used in quantity sufficient to make different volumes for different strengths.

In another aspect of the invention, the compositions may be prepared with or without Polysorbate 80, with or without Monothioglycerol.

The present invention provides stable aqueous liquid injectable composition of Methisoprinol comprising upto 150 mg/ml fit for administration via multiple routes such as IM, IV and infusion. Composition of the present invention can be filled in vials and ampoules.

In another embodiment, the injectable composition may be filled in suitable containers such as ampoule and vials or PFS or special syringes like BDS with specific volume and placed in pouch or filled in smaller canisters, filled in containers for use as spray, nasal drops, otic drops, delivered in devices used for brain drug delivery after aseptic filtration and flushed under nitrogen blanket or terminally sterilized. The composition may be available as single use or multiple use vials or glass ampoules or glass ampoules with black ring or with non-reactive glass ampoules or filled in bags for LVP using diluents and buffers or ready to infuse kit such as bags and glass or plastic bottles preferably of single compartment which may be composed of low density polyethylene (LDPE), high density polyethylene (HDPE), polypropylene (PP) or mixtures of polyethylene and polypropylene. Glass bottles in packaging of said composition are composed of mixture of crystalline oxides and carbonates. Preferably used glass material is USP type I glass (borosilicate glass) and type II glass (soda lime glass with chemical surface treatment). In another embodiment, the said composition may be provided in volume of equal or greater than 1 ml to 500 ml of single compartment depending upon quantity of Methisoprinol dissolved per ml of solution or ready-to-use injection which can be diluted according to physiological use. Further the kit said herein may be also of flexible bags.

In another embodiment, closures used may include sealing or rubber stoppers, closures or disc seals, screw-caps or cap-stopper combination seals closures may be used as a component of packaging.

As aqueous Methisoprinol composition of the present invention is stable, it can as well be filled in glass bottles and supplied for oral consumption with or without flavours and sweeteners.

The composition may be sterilized by aseptic filtration suitable for sterilization. The formulation is prepared and filled in article. Sterilization can be attained by electron beam irradiation, γ-irradiation, natural light, microwave heat viz. moist heat sterilization or terminal sterilization or aseptic filtration.

The injectable compositions so prepared are stable and may be diluted further in infusion liquid to obtain the desired strength of drug. The injectable composition can be administered by intramuscular and as slow bolus intravenous or suitably adding to infusion liquid as per physician need.

Some of the examples for formulations prepared in accordance with the present invention and the process to prepare the compositions of the present inventions are as given in the examples section. These stable aqueous compositions are filled in ampoules or vials or any other suitable containers fit for use as per known practices in the pharmaceutical production processes, to enable desired administration.

Compositions envisaged, claimed and covered by this invention include all compositions comprising concentrations of Methisoprinol upto 150 mg/ml in different concentrations and expressed in different volumes. Thus 50 mg/0.5 ml or 50 mg/ml fall within the ambit of present invention. Similarly concentrations of 25 mg or 75 mg/ml fall within the ambit of the present invention whether present in 1 ml or in volume less than 1 ml and upto 1 ml such as 0.25 ml or 0.5 ml. Similarly Methisoprinol compositions having 100 mg/ml or 150 mg/ml fall within the ambit of present invention.

Compositions provided by present inventions are aqueous compositions comprising water as a solvent in presence of an alcohol selected from monohydric or polyhydric or mixture of two. When more than one solvent is used at least one of them is to be interpreted as co-solvent.

Batches of composition of the formula as depicted in Example 1 were prepared as per the procedure as depicted in Example 2 and all were found to be stable for at least 6 months at 40° C./75% RH, 25° C./60% RH, 30° C./75% RH, 30° C./65% RH when subjected to stability studies.

As described hereinbefore, the term stable is to be interpreted as stable under 30° C./75% RH, 30° C./65% RH, 40° C./75% RH, 25° C./60% RH and 2° C.-8° C. conditions. Stabilization refers to solubilization or dissolving, keeping the active in dissolved state and keeping active content in acceptable specified limits. Stable composition also refers to a composition wherein even if any crystallization takes place during storage, crystals re-dissolve into the solution on shaking.

The above and other features and advantages of the present invention will now be described with reference to the following examples, which are given by way of illustration and should not be construed to limit the scope of the present invention.

While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims. An additional active ingredient may also be incorporated in the composition provided by the present invention.

### EXAMPLES

### Example 1: Composition Comprising 100 mg/ml Methisoprinol

TABLE 1

| | | Standard | |
|---|---|---|---|
| | | Quantity/ | |
| Item Name | Qty./mL | Batch | UOM |
| Methisoprinol ((Inosine | 10% w/v | 2.50 | Kg |
| Pranobex (Inosine Dimepranol | (100.0 mg) | | |
| Acedoben) IH) | | | |
| Propylene glycol IP | 15% w/v | 3.75 | Kg |
| | (150.0 mg) | | |
| Glycerin IP | 15% w/v | 3.75 | Kg |
| | (150.0 mg) | | |
| Monothioglycerol USP-NF | 0.25% w/V | 0.063 | Kg |
| | (2.5mg) | | |
| Sodium chloride IP | 0.9% w/v | 0.225 | Kg |
| | (9.0 mg) | | |
| Water for injection IP | q.s. to | q.s. to 25 | L |
| | 1 mL | | |
| Packing Materials | | | |
| 10 mL clear ampoule with | — | 2548 | Nos. |
| white snap off ring USP Type I | | | |

The above composition was packed in vial and labeled as 1000 mg/10 mL Batch No INN001.

The above composition was packed in ampoule and labeled as 1000 mg/10 mL Batch No INE001.

Quantity of Methisoprinol is given on the basis 100% assay and 0% water content. Approximately 5% extra primary packing material is considered. Similar volume was packed as follows:

| Packing Materials | Quantity | UOM |
|---|---|---|
| 13 mm 10 mL Clear Vial USP Type I | 2548@ | Nos. |
| 13 mm bromo butyl rubber plug | 2548@ | Nos. |
| 13 mm Plastic Flip Off Seal Green Color | 2548@ | Nos. |

### Example 2: Process to Prepare Methisoprinol Aqueous Liquid Composition

The composition of Example 1 was prepared by following process. All the ingredients were added according to the amounts provided in table 1.
1. All the ingredients as mentioned in table 1 were weighed by suitable means.
2. In jacketed manufacturing tank about 30 liters of water was collected for injection (WFI) and cooled to 25±2° C., with continuous stirring and nitrogen bubbling/sparging, in closed condition. About 20 liters WFI was transferred from above jacketed manufacturing tank to separate SS vessel and nitrogen bubbling/sparging was started. The remaining water was kept aside for volume adjustment.
3. Sodium chloride was added in above jacketed manufacturing tank under stirring and nitrogen bubbling/sparging. The stirring was performed for 5 minutes with lid of the tank remained closed.
4. Propylene glycol, glycerin and monothioglycerol were added into the jacketed manufacturing tank under stirring and nitrogen bubbling/sparging. The lid was closed and stirring was performed till a clear solution was obtained.
5. Methisoprinol was added into the jacketed manufacturing tank under stirring and nitrogen bubbling/sparging. The lid was closed and stirring was performed till a clear solution was obtained.
6. The pH of the clear solution obtained in above step was adjusted to 6.0 to 7.5.
7. The final volume was made to 25 liters by adding WFI from the SS container. The solution was stirred for about 30 minutes with nitrogen bubbling/sparging in a closed tank till a clear solution was formed.
8. All the openings of jacketed manufacturing tank were closed. Blanketed the manufacturing tank with nitrogen bubbling till filtration was complete.

### First Filtration Procedure

1. Sterile filter assembly was arranged and the filter with water for injection was wetted.
2. The pre bubble point test was performed for 0.22μ Nylon 66 membrane filters.
3. The sterilized assembly was arranged for filtration. The pressure vessel was pre flushed with sterile nitrogen prior to start of filtration.
4. Filtered through 2.0μ and 0.22μ membrane filter with help of nitrogen.
5. After completion of filtration, the post bubble point test was performed.

### Second Filtration Procedure (Before Filling)

1. Sterile filter assembly was arranged and the filter with water for injection was wetted.
2. The pre bubble point test was performed for 0.22μ Nylon 66 membrane filters.
3. The sterilized assembly was arranged for filtration. The pressure vessel was pre flushed with sterile nitrogen prior to start of filtration.
4. Filtration was carried through 0.22μ membrane filter with help of filtered nitrogen and solution was collected in filling vessel.
5. The filtration was started along with continuous filling activity.
6. After completion of filtration, the post bubble point test was performed.

The aqueous injectable solutions of Methisoprinol thus prepared were filled into suitable containers such as vials and ampoules, sealed and subjected to stability testing under different conditions.

### Example 3: Stability Assessment of Compositions of Present Invention

A: Stability Assessment of Composition of Example 1, when Packed in Vial [Batch No INN001]

Stability of composition of batch INN001 was assessed at 40° C.±2° C./75% RH±5% RH after six months. The results are presented in the Table 2.

TABLE 2

| Sr. | | |
|---|---|---|
| No | Test: Specification | 6M |
| 1. | Description: Clear colourless solution | Clear |
| | | colourless |
| | | solution |
| 2. | pH: Between 6.0 and 7.5 | 6.75 |
| 3 | Related substances (Chromatogram purity ) (BY HPLC) | |
| 3.1 | Hypoxanthine: NMT 0.2% | ND |
| 3.2 | 4-Aminobenzoic acid: NMT 0.2% | 0.07% |
| 3.3 | Single unspecified impurity: NMT 0.1% | ND |
| 3.4 | Sum of Impurities: NMT 0.5% | 0.07% |
| | Assay (By HPLC) | |
| 41 | Inosine Pranobex: NLT95.0% and NMT 105.0% of | 100.05% |
| | label claim. (950.0 mg to 1050.0 mg) | |
| 4.2 | Inosine: 240 mg/10 ml.(228.0 mg to 252.0 mg) | 100.85% |
| | (NLT 95.0% to NMT 105.0%) | |
| 4.3 | 4-Acetamedobenzoic acid salt with N,N-dimethlamino | 99.80% |
| | 2-propanol: 760 mg/10 ml. (722.0 mg to 798.0 mg). | |
| | (NLT 95.0% to NMT 105.0%) | |

* NMT is “not more than” and NLT is “not less than”

The batch was also subjected to tests of bacterial endotoxin, sterility and particulate matter. The batch conforms to sterility standards as per ICH guidelines.

The composition of Example 1 filled and sealed in vial as well as ampoule, was found stable after 6 months testing under accelerated and room temperature conditions.

B: Stability Assessment of Composition of Example 1, when Packed in Ampoule [Batch No INE001]

Batch No INE001 packed in ampoule was stable at 40° C.±2° C./75%±5% RH, 25° C.±2° C./60%±5% RH, 30° C.±2° C./65%±5% RH, 6 months stability testing results at 40° C.±2° C./75%±5% RH are as follows:

TABLE 3

| Sr. | | |
|---|---|---|
| No | Test: Specification | 6M |
| 1. | Description: Clear colourless solution | Clear |
| | | colourless |
| | | solution |
| 2. | pH: Between 6.0 and 7.5 | 6.65 |
| 3 | Related substances (Chromatogram purity ) (BY HPLC) | |
| 3.1 | Hypoxanthine: NMT 0.2% | ND |
| 3.2 | 4- Aminobenzoic acid: NMT 0.2% | 0.12% |
| 3.3 | Single unspecified impurity: NMT 0.1% | ND |
| 3.4 | Sum of Impurities: NMT 0.5% | 0.12% |
| | Assay (By HPLC) | |
| 41 | Inosine Pranobex: NLT95.0% and NMT 105.0% | 100.60% |
| | label claim. (950.0 mg to 1050.0 mg) | |
| 4.2 | Inosine: 240 mg/10 ml.(228.0 mg to 252.0 mg) | 99.67% |
| | (NLT 95.0% to NMT 105.0%) | |
| 4.3 | 4-Acetamedobenzoic acid salt with N,N-dimethlamino | 100.60% |
| | 2-propanol: 760 mg/10 ml. (722.0 mg to 798.0 mg). | |
| | (NLT 95.0% to NMT 105.0%) | |

* NMT is “not more than” and NLT is “not less than”

This batch also conformed to bacterial endotoxin and sterility test specifications.

### Example 4: Methisoprinol Composition without Preservative

A Methisoprinol composition without preservative was prepared by employing ingredients as depicted in Table 4 below.

TABLE 4

| Ingredients | IPI-2019 |
|---|---|
| Methisoprinol ((Inosine | 100 |
| Pranobex (Inosine | |
| Dimepranol Acedoben) IH) | |
| (mg/mL) | |
| Propylene glycol (% w/v) | 15.0 |
| Glycerin (% w/v) | 15.0 |
| Acetate Buffer | qs to 1 ml |

### Example 5: Process to Prepare Methisoprinol Aqueous Liquid Composition of Example 4

The Methisoprinol composition of Example 4 was prepared by the process comprising the following steps:
1. Methisoprinol was added to propylene glycol and glycerin and stirred to dissolve. Volume was adjusted by acetate buffer.
2. Composition obtained in step 1 was filtered.
3. The filtered composition obtained in step 2 was filled and sealed in suitable containers such as vials.
4. Filled vials obtained in step 3 were subjected to stability testing at 40° C./75% RH, 30° C./75% RH, 25° C./60% RH and 2° C.-8° C. These were not sterile samples. They were stable.

### Example 6: Stability Assessment of Composition of Example 4

Stability of composition of Example 4 was assessed after 30 days. The results are presented in the Table 5.

TABLE 5

| Description | Clear colorless solution |
|---|---|
| pH | 6.30-6.37 |
| Inosine Pranobex Assay (95.0%-105.0%) | 99.45%-99.90% |
| Inosine Assay- NLT 95.0% to NMT 105.0% | 96.97%-97.46% |
| Acetamidobenzoic acid Assay - NLT 95.0% | 100.23%-102.67% |
| to NMT 105.0% | |

* NMT is “not more than” and NLT is “not less than”

### Example 7: Compositions Comprising 125 mg/ml Methisoprinol (Batch IPI-2025 and Batch IPI-2026)

A Methisoprinol composition comprising 125 mg/ml of Methisoprinol was prepared by employing ingredients as depicted in Table 6 below.

TABLE 6

| Ingredients | IPI-2025 | IPI-2026 |
|---|---|---|
| Methisoprinol ((Inosine Pranobex | 125 mg | 125 mg |
| (Inosine Dimepranol Acedoben) | | |
| IH) (mg/mL) | | |
| Sodium chloride (% w/v) | 0.90 | 0.90 |
| Propylene glycol (% w/v) | NA | 20.00 |
| Monothioglycerol (% w/v) | 0.25 | 0.25 |
| Glycerin (% w/v) | 40.00 | 20.00 |
| Water for injection | q.s to 1 mL | q.s to 1 mL |

The compositions were prepared according to the process explained in Example 2. As may be seen from table 6, one composition (IPI-2025) was prepared by using propylene glycol and glycerin. Second composition (IPI-2026) was prepared by using only glycerin. The propylene glycol was not used in the second composition. The compositions IPI-2025 and IPI-2026 were subjected to 2° C.-8° C. stability studies. The pH, Inosine and 4-acetamidobenzoic acid were well within the range or acceptance criteria. The impurities were within the limit and there was no crystallization. The compositions were stable.

### Example 8: Composition Comprising 150 mg/ml Methisoprinol (Batch IPI-2027)

A Methisoprinol composition comprising 150 mg/ml of Methisoprinol was prepared by employing ingredients as depicted in Table 7 below.

TABLE 7

| Ingredients | IPI-2027 |
|---|---|
| Methisoprinol ((Inosine Pranobex | 150 mg |
| (Inosine Dimepranol Acedoben) | |
| IH) (mg/mL) | |
| Sodium Chloride (% w/v) | 0.90 |
| Monothioglycerol (% w/v) | 0.25 |
| Glycerin (% w/v) | 20.00 |
| Water for Injection | q.s to 1 mL |

The composition was prepared according to the process explained in Example 2. Samples of Batch IPI-2027 were subjected to 2° C.-8° C. stability studies for 18 days. pH, assay of inosine and 4-acetamidobenzoic acid was well within the range acceptance criteria and impurities were within the limits as shown in Example 3. Mild crystallization was observed but on mild shaking crystals dissolved in bulk and there was ‘a clear colorless solution’ in the container. The composition was stable.

### Example 9: Composition Comprising 100 mg/ml Methisoprinol (Batch IPI-2014)

A Methisoprinol composition comprising 100 mg/ml of Methisoprinol was prepared by employing ingredients as depicted in Table 8 below.

TABLE 8

| Ingredients | IPI-2014 |
|---|---|
| Methisoprinol ((Inosine Pranobex | 100.00 |
| (Inosine Dimepranol Acedoben) | |
| IH) (mg/mL) | |
| Sodium Chloride (% w/v) | 0.90 |
| Propylene glycol (% w/v) | 10.00 |
| Sodium Metabisulphite (% w/v) | 0.1 |
| Ethanol (% w/v) | 10.00 |
| Water for Injection | q.s to 1 mL |
| Batch size | 100 ml |

A non-sterile composition stated above was stable at 40° C. for 3 months. No crystals observed. Assay and impurities were well within the limits.

### Manufacturing Procedure:

1. Collected about 50 mL of water for injection (WFI) in a glass beaker.
2. Added dispensed quantity of sodium chloride and Sodium Metabisulphite, in above glass beaker and rinsed container two times with WFI under stirring and nitrogen bubbling/sparging, overlaying. Stirred to mix for about 5 minutes.
3. Added dispensed quantity of propylene glycol and Ethanol in above glass beaker. Stirred to get a clear solution.
4. Added dispensed quantity of Inosine Pranobex in above glass beaker. Stirred to get a clear solution.
5. Checked pH of solution of jacketed manufacturing tank and it was between 6.0 to 7.5.
6. Made up the final volume to 100 mL by WFI. Stirred the solution for about 30 minutes with nitrogen bubbling/sparging and overlaying. Stopped the stirring after completion of time. Checked clarity of solution.
7. Collected the sample of bulk solution into glass vial and sent it for bulk analysis. The dispensed quantity mentioned above refers to the amounts disclosed in Table 8.

Similarly compositions were prepared by taking proportions of propylene glycol:ethyl alcohol in the ratio of 1:10 to 10:1. These compositions can be prepared with or without incorporating glycerin. Similarly compositions were prepared where propylene glycol was replaced by glycerin. However, for the reason of brevity of, and conciseness of specification such compositions have not been illustrated herein, as these are easy to prepare using same procedure.

### Example 10: Stability Assessment of Composition of Example 9

Results of stability study for 2 months (2M) of batch number depicted in Example 9 are as follows:

TABLE 9

| | | | 40° C./ | 25° C./ | 2° C.- |
|---|---|---|---|---|---|
| Sr. | | | 75% RH | 60% RH | 8° C. |
| No | Test | Initial | 2M | 2M | 2M |
| 1 | Description | A Clear | A Clear | A Clear | A Clear |
| | | colorless | colorless | colorless | colorless |
| | | solution | solution | solution | solution |
| 2 | pH (at 25° C.) | 5.76 | 5.8 | 5.71 | 5.67 |
| | 5.0 to 7.0 | | | | |
| 3 | Inosine Pranobex | 99.86% | 100.87% | 100.16% | 100.68% |
| | 100 mg | | | | |
| | (NLT 95.0% and | | | | |
| | NMT 105.0%) | | | | |
| | Inosine + 4- | | | | |
| | Acetamidobenzoic | | | | |
| | acid | | | | |
| | Assay: Inosine- | 97.99% | 99.66% | 99.12% | 99.60% |
| | 24 mg | | | | |
| | (NLT 95.0% and | | | | |
| | NMT 105.0%) | | | | |
| | Assay: 4- | 100.45% | 101.25% | 100.49% | 101.03% |
| | Acetamidobenzoic | | | | |
| | acid - 76 mg | | | | |
| | (NLT 95.0% and | | | | |
| | NMT 105.0%) | | | | |

* NMT is “not more than” and NLT is “not less than”.

### Example 11: Stability Assessment of Batch No INN002

Batch No INN002 was produced in identical manner as that of INN001 and packed in vial. Stability study was performed at 25° C.±2° C./60%±5% RH for 6 months (6M). Results of the same are given below:

TABLE 10

| SrNo | Test: Specification. | 6M |
|---|---|---|
| 1. | Description: Clear colourless solution | Clear |
| | | colourless |
| | | solution |
| 2. | pH: Between 6.0 and 7.5 | 6.75 |
| 3 | Related substances (Chromatogram purity ) (BY HPLC) | |
| 3.1 | Hypoxanthine: NMT 0.2% | 0.08% |
| 3.2 | 4-Aminobenzoic acid: NMT 0.2% | ND |
| 3.3 | Single unspecified impurity: NMT 0.1% | ND |
| 3.4 | Sum of Impurities: NMT 0.5% | 0.08% |
| 4 | Assay (By HPLC) | |
| 4.1 | Inosine Pranobex: NLT95.0% and NMT 105.0% | 100.63% |
| | of label claim, (950.0 mg to 1050.0 mg) | |
| 4.2 | Inosine: 240 mg/10 ml. (228.0 mg to 252.0 mg) | 101.7% |
| | (NLT 95.0% to NMT 105.0%) | |
| 4.3 | 4-Acetamedobenzoic acid salt with N,N-dimethlamino- | 100.30% |
| | 2-propanol: 760 mg/10 ml. (722.0 mg to 798.0 mg) | |
| | (NLT 95.0% to NMT 105.0%) | |

Batch INN002 also conformed to bacterial endotoxin and sterility test specifications and hence confirms reproducibility of the composition of present invention

### Example 12: Comparative Example

One litre compositions of 100 mg/ml and 150 ml/ml of Methisoprinol were prepared having alcohol content of 500 and 1000 w/v. The per ml compositions of the same are depicted in tables 11 and 12 below:

TABLE 11

| | TIP- | TIP- | TIP- | TIP- |
|---|---|---|---|---|
| Ingredients | 2011 | 2012 | 2013 | 2014 |
| Methisoprinol (mg/mL) | 150 mg | 100 mg | 150 mg | 100 mg |
| Sodium Chloride (% w/v) | 0.90 | 0.90 | 0.90 | 0.90 |
| Monothioglycerol (% w/v) | 0.25 | 0.25 | 0.25 | 0.25 |
| Ethyl Alcohol (% w/v) | 10.00 | 10.00 | 5.00 | 5.00 |
| Propylene Glycol (% w/v) | — | — | — | — |
| Water for Injection | q.s to | q.s to | q.s to | q.s to |
| | 1 mL | 1 mL | 1 mL | 1 mL |

TABLE 12

| | TIP- | TIP- | TIP- | TIP- |
|---|---|---|---|---|
| Ingredients | 2015 | 2016 | 2017 | 2018 |
| Methisoprinol (mg/mL) | 150 mg | 100 mg | 150 mg | 100 mg |
| Sodium Chloride (% w/v) | 0.90 | 0.90 | 0.90 | 0.90 |
| Monothioglycerol (% w/v) | 0.25 | 0.25 | 0.25 | 0.25 |
| Ethyl Alcohol (% w/v) | — | — | — | — |
| Propylene Glycol (% w/v) | 10 | 10 | 5 | 5 |
| Water for Injection | q.s to | q.s to | q.s to | q.s to |
| | 1 mL | 1 mL | 1 mL | 1 mL |

Compositions of 100 mg and 150 mg of Methisoprinol per ml having alcohol content 500 and 1000 as depicted in tables above were not stable.

Other embodiments of the invention will be apparent to those skilled in the art from consideration of the specification and practice of the embodiments disclosed herein. It is intended that the specification and examples be considered as exemplary only, with a true scope and spirit of the invention being indicated by the following claims. In addition, where this application has listed the steps of a method or procedure in a specific order, it may be possible, or even expedient in certain circumstances, to change the order in which some steps are performed, and it is intended that the particular steps of the method or procedure claims set forth herein below not be construed as being order-specific unless such order specificity is expressly stated in the claim.

1. A stable aqueous composition comprising Methisoprinol in an amount from 25 mg/ml to 150 mg/ml, at least one alcohol in an amount from 20% w/v to 40% w/v and at least one excipient selected from pH modifier or buffer, surfactant, solvents, co-solvent, preservative, antioxidants, chelating agent and tonicity modifier.

2. The composition as claimed in claim 1 wherein composition comprises 100 mg/ml to 150 mg/ml methisoprinol and 30% w/v to 40% w/v alcohol.

3. The composition as claimed in claim 1 wherein the composition comprises alcohol selected from monohydric alcohol such as ethyl alcohol, benzyl alcohol or polyhydric alcohol such as glycerin, propylene glycol, monothioglycerol, sorbitol or combination thereof.

4. The composition as claimed in claim 3, wherein the glycerin is present in an amount between 1% w/v to 40% w/v, preferably between 15% w/v to 40% w/v.

5. The composition as claimed in claim 3, wherein the propylene glycol is present in an amount between 10% w/v to 20% w/v.

6. The composition as claimed in claim 3, wherein ethyl alcohol is present in in an amount between 10% w/v to 20% w/v.

7. The composition as claimed in claim 3, wherein the composition comprises propylene glycol and glycerin in a ratio from 0.0:40 to 3:1, preferably from 0.0:40 to 1:1.

8. The composition as claimed in claim 3, wherein the composition comprises ethyl alcohol and glycerin in a ratio of 0:10 to 10:0, preferably 1:1.

9. The composition as claimed in claim 3, wherein the composition comprises ethyl alcohol and propylene glycol in the ratio from 1:10 to 10:1.

10. The composition as claimed in claim 1, wherein the composition comprises 100 mg/ml Methisoprinol, 20% w/v propylene glycol and 20% w/v glycerin.

11. The composition as claimed in claim 1, wherein the composition comprises 100 mg/ml Methisoprinol, 15% w/v propylene glycol and 15% w/v glycerin.

12. The composition as claimed in claim 1, wherein the composition comprises 150 mg/ml Methisoprinol, 20% w/v propylene glycol and 20% w/v glycerin.

13. The composition as claimed in claim 1, wherein the composition comprises 125 mg/ml Methisoprinol and 20% w/v to 40% w/v glycerin.

14. The composition as claimed in claim 10, wherein the composition further comprises monothioglycerol in an amount from 0.25% w/v to 1% w/v and sodium chloride up to 1% w/v.

15. A stable, clear, aqueous composition comprising 100 mg/ml Methisoprinol, 20% w/v propylene glycol and 20% w/v glycerin, 0.25% w/v monothioglycerol and 1% w/v sodium chloride.

16. The composition of claim 1, wherein the composition is an injectable composition and is suitable for parenteral administration.

17. The composition of claim 1, wherein the composition having viscosity not more than 10 cps.

18. The composition of claim 1, wherein the composition has a pH from 6.0 to 7.5.

19. (canceled)

20. A method of preparing a stable aqueous composition comprising Methisoprinol, wherein the method comprises:
a) weighing required quantities of all raw materials by suitable means;
b) collecting sufficient quantity of water in jacketed manufacturing tank and cooling it to about 25±2° C. with continuous stirring and nitrogen sparging in closed condition;
c) transferring about ⅔rd of water for injection from jacketed manufacturing tank referred in step b) to separate SS vessel and starting nitrogen bubbling while keeping remaining quantity of water aside for volume adjustment;
d) adding required quantity of sodium chloride in jacketed manufacturing tank referred in b) under stirring and nitrogen bubbling followed by stirring for about 5 minutes with lid closed;
e) adding required quantity of alcohol into the jacketed manufacturing tank referred in b) under stirring and nitrogen bubbling followed by optionally adding one or more stabilizers under continuous stirring till a clear solution is obtained;
f) adding required quantity of Methisoprinol to clear solution obtained in step (e) under stirring and nitrogen bubbling while keeping the tank closed under continuous stirring to obtain a clear solution;
g) adjusting pH of the clear solution obtained in step (f) to about 6.0 to 7.5;
h) adding water to make final volume while continuously stirring for about 30 minutes with nitrogen bubbling in a closed tank until clear solution was obtained;
i) closing all openings of the jacketed manufacturing tank blanketed with nitrogen bubbling until filtration is complete;
j) optionally, packing the solution prepared in step (i) after filtration in suitable containers.

21-22. (canceled)

23. A method of treatment or prevention of viral disease and immune-suppressed states comprising administering to a patient in need thereof, a therapeutically effective amount of the composition of claim 1.

24. The method as claimed in claim 23, wherein the viral disease is selected from the group consisting of genital warts, subacute sclerosingpanencephalitis (SSPE), herpes simplex virus (HSV) and varicella infections, human papilloma virus (HPV), cytomegalovirus and Epstein-Barr virus infections, acute viral respiratory infections, exanthematous viral infections like chickenpox, measles, bronchitis, common cold (rhinopharyngitis).
